(19) United States
(12) Patent Application Publication
(10) Pub. No.: US 2003/0203384 A1
(43) Pub. Date: Oct. 30, 2003

(54) Multiplex detection of biological materials in a sample

(76) Inventors: David R. Chafin, Rochester, NY (US); Dennis M. Connolly, Rochester, NY (US)

Correspondence Address: Michael L. Goldman, NIXON PEABODY LLP, Clinton Square, P.O. Box 31051, Rochester, NY, 14603-1051, US

(21) Appl. No.: 10/383,397

(22) Filed: Mar. 6, 2003

Related U.S. Application Data
(60) Provisional application No. 60/363,348, filed on Mar. 8, 2002.

(51) Int. Cl. C12Q 1/68; C12M 1/34

(52) U.S. Cl. 435/6; 435/287.2

(57) ABSTRACT

The present invention relates to a method of detecting target nucleic acid molecules in a sample. This method involves providing a plurality of different groups of two or more electrically separated electrical conductors with capture probes attached to the conductors such that a gap exists between the capture probes on the electrically separated conductors. The capture probes are contacted with a sample, potentially containing the target nucleic acid molecules, under conditions effective to permit any of the target nucleic acid molecule present in the sample to hybridize to the capture probes and thereby connect the capture probes. The presence of the target nucleic acid molecules is detected by determining whether electricity is conducted between the electrically separated conductors. Devices for carrying out this method are also disclosed.

This application claims the benefit of U.S. Provisional Patent Application Serial No. 60/363,348, filed Mar. 8, 2002.

### FIELD OF THE INVENTION

The present invention relates to the multiplex detection of target molecules, such as deoxyribonucleic acids (DNA) or ribonucleic acids (RNA), from fluid samples.

### BACKGROUND OF THE INVENTION

Nucleic acids, such as DNA or RNA, have become of increasing interest as analytes for clinical or forensic uses. Powerful new molecular biology technologies enable one to detect congenital or infectious diseases. These same technologies can characterize DNA for use in settling factual issues in legal proceedings, such as paternity suits and criminal prosecutions.

For the analysis and testing of nucleic acid molecules, amplification of a small amount of nucleic acid molecules, isolation of the amplified nucleic acid fragments, and other procedures are necessary. The science of amplifying small amounts of DNA have progressed rapidly and several methods now exist. These include linked linear amplification, ligation-based amplification, transcription-based amplification and linear isothermal amplification. Linked linear amplification is described in detail in U.S. Pat. No. 6,027,923 to Wallace et al. Ligation-based amplification includes the ligation amplification reaction (LAR) described in detail in Wu et al., Genomics, 4:560 (1989) and the ligase chain reaction described in European Patent No. 0320308B1. Transcription-based amplification methods are described in detail in U.S. Pat. Nos. 5,766,849 and 5,654,142, Kwoh et al., Proc. Natl. Acad. Sci. U.S.A., 86:1173 (1989), and PCT Publication No. WO 88/10315 to Ginergeras et al. The more recent method of linear isothermal amplification is described in U.S. Pat. No. 6,251,639 to Kurn.

The most common method of amplifying DNA is by the polymerase chain reaction (“PCR”), described in detail by Mullis et al., Cold Spring Harbor Quant. Biol., 51:263-273 (1986), European Patent No. 201,184 to Mullis, U.S. Pat. No. 4,582,788 to Mullis et al., European Patent Nos. 50,424, 84,796, 258017, and 237362 to Erlich et al., and U.S. Pat. No. 4,683,194 to Saiki et al. The PCR reaction is based on multiple cycles of hybridization and nucleic acid synthesis and denaturation in which an extremely small number of nucleic acid molecules or fragments can be multiplied by several orders of magnitude to provide detectable amounts of material. One of ordinary skill in the art knows that the effectiveness and reproducibility of PCR amplification is dependent, in part, on the purity and amount of the DNA template. Certain molecules present in biological sources of nucleic acids are known to stop or inhibit PCR amplification (Belec et al., Muscle and Nerve, 21(8):1064 (1998); Wiedbrauk et al., Journal of Clinical Microbiology, 33(10):2643-6 (1995); Deneer and Knight, Clinical Chemistry, 40(1):171-2 (1994)). For example, in whole blood, hemoglobin, lactoferrin, and immunoglobulin G are known to interfere with several DNA polymerases used to perform PCR reactions (Al-Soud and Radstrom, Journal of Clinical Microbiology, 39(2):485-493 (2001); Al-Soud et al., Journal of Clinical Microbiology, 38(1):345-50 (2000)). These inhibitory effects can be more or less overcome by the addition of certain protein agents, but these agents must be added in addition to the multiple components already used to perform the PCR. Thus, the removal or inactivation of such inhibitors is an important factor in amplifying DNA from select samples.

On the other hand, isolation and detection of particular nucleic acid molecules in a mixture requires a nucleic acid sequencer and fragment analyzer, in which gel electrophoresis and fluorescence detection are combined. Unfortunately, electrophoresis becomes very labor-intensive as the number of samples or test items increases.

For this reason, a simpler method of analysis using DNA oligonucleotide probes is becoming popular. New technology, called VLSIPS™, has enabled the production of chips smaller than a thumbnail where each chip contains hundreds of thousands or more different molecular probes. These techniques are described in U.S. Pat. No. 5,143,854 to Pirrung et al., PCT Publication No. WO 92/10092, and PCT WO 90/15070. These biological chips have molecular probes arranged in arrays where each probe ensemble is assigned a specific location. These molecular array chips have been produced in which each probe location has a center to center distance measured on the micron scale. Use of these array type chips has the advantage that only a small amount of sample is required, and a diverse number of probe sequences can be used simultaneously. Array chips have been useful in a number of different types of scientific applications, including measuring gene expression levels, identification of single nucleotide polymorphisms, and molecular diagnostics and sequencing as described in U.S. Pat. No. 5,143,854 to Pirrung et al.

Array chips where the probes are nucleic acid molecules have been increasingly useful for detection for the presence of specific DNA sequences. Most technologies related to array chips involve the coupling of a probe of known sequence to a substrate that can either be structural or conductive in nature. Structural types of array chips usually involve providing a platform where probe molecules can be constructed base by base or covalently binding a completed molecule. Typical array chips involve amplification of the target nucleic acid followed by detection with a fluorescent label to determine whether target nucleic acid molecules hybridize with any of the oligonucleotide probes on the chip. After exposing the array to a sample containing target nucleic acid molecules under selected test conditions, scanning devices can examine each location in the array and quantitate the amount of hybridized material at that location. Alternatively, conductive types of array chips contain probe sequences linked to conductive materials such as metals. Hybridization of a target nucleic acid typically elicits an electrical signal that is carried to the conductive electrode and then analyzed.

For most solid support or array technologies, small oligonucleotide capture probes are immobilized or synthesized on the support. The sequence of the capture probes imparts the specificity for the hybridization reaction. Several different chemical compositions exist currently for capture probe studies. The standard for many years has been straight deoxyribonucleic acids. The advantage of these short single stranded DNA molecules is that the technology has existed for many years and the synthesis reaction is relatively inexpensive. Furthermore, a large body of technical studies is available for quick reference for a variety of scientific techniques, including hybridization. However, many different types of DNA analogs are now being synthesized commercially that have advantages over DNA oligonucleotides for hybridization. Some of these include PNA (protein nucleic acid), LNA (locked nucleic acid) and methyl phosphonate chemistries. In general, all of the DNA analogs have higher melting temperatures than standard DNA oligonucleotides and can more easily distinguish between a fully complementary and single base mis-match target. This is possible because the DNA analogs do not have a negatively charged backbone, as is the case with standard DNA. This allows for the incoming strand of target DNA to bind tighter to the DNA analog because only one strand is negatively charged. The most studied of these analogs for hybridization techniques is the PNA analog, which is composed of a protein backbone with substituted nucleobases for the amino acid side chains (see www.appliedbiosystems.com or www.eurogentec.com). Indeed, PNAs have been used in place of standard DNA for almost all molecular biology techniques including DNA sequencing (Arlinghaus et al., Anal Chem., 69:3747-53 (1997)), DNA fingerprinting (Guerasimova et al., Biotechniques, 31:490-495 (2001)), diagnostic biochips (Prix et al., Clin. Chem., 48:428-35 (2002); Feriotto et al., Lab Invest, 81:1415-1427 (2001)), and hybridization based microarray analysis (Weiler et al., Nucleic Acids Res, 25:2792-2799 (1997); Igloi, Genomics, 74:402-407 (2001)).

Techniques for forming sequences on a substrate are known. For example, the sequences may be formed according to the techniques disclosed in U.S. Pat. No. 5,143,854 to Pirrung et al., PCT Publication No. WO 92/10092, or U.S. Pat. No. 5,571,639 to Hubbell et al. Although there are several references on the attachment of biologically useful molecules to electrically insulating surfaces such as glass (http://www.piercenet.com/Technical/default.cfin?tmpl=../Lib/ViewDoc.cfm&doc=3483; McGovern et al., Langmuir, 10:3607-3614 (1994)) or silicon oxide (Examples 4-6 of U.S. Pat. No. 6,159,695 to McGovern et al.), there are few examples of effective molecular attachment to electrically conducting surfaces except for gold (Bain et al., Langmuir, 5:723-727 (1989)) and silver (Xia et al., Langmuir, 22:269, (1998)). In general, the problem of attaching biologically active molecules to the surface of a substrate, whether it is a metal electrical conductor or an electrical insulator such as glass, is more difficult than the simple chemical reaction of a reactive group on the biological molecule with a complementary reactive group on the substrate. For example, a metal electrical conductor has no reactive sites, in principle, except those that may be adventitiously or deliberately positioned on the surface of the metal.

Hybridization of target DNAs to such surface bound capture probes poses difficulties not seen, if both species are soluble. Steric effects result from the solid support itself and from too high of a probe density. Studies have shown that hybridization efficiency can be altered by the insertion of a linker moiety that raises the complementary region of the probe away from the surface (Schepinov et al., Nucleic Acid Res., 25:1155-1161 (1997); Day et al., Biochem J., 278:735-740 (1991)), the density at which probes are deposited (Peterson et al., Nucleic Acids Res., 29:5163-5168 (2001); Wilkins et al., Nucleic Acids Res., 27:1719-1729)), and probe conformation (Riccelli et al., Nucleic Acids Res., 29:996-1004 (2001)). Insertion of a linker moiety between the complementary region of a probe and its attachment point can increase hybridization efficiency and optimal hybridization efficiency has been reported for linkers between 30 and 60 atoms in length. Likewise, studies of probe density suggest that there is an optimum probe density, and that this density is less than the total saturation of the surface (Schepinov et al., Nucleic Acid Res., 25:1155-1161 (1997); Peterson et al., Nucleic Acids Res., 29:5163-5168 (2001); Steel et al., Anal. Chem., 70:4670-4677 (1998)). For example, Peterson et al. reported that hybridization efficiency decreased from 95% to 15% with probe densities of 2.0×10¹² molecules/cm² and 12.0×10¹² molecules/cm², respectively.

Quantitiation of hybridization events often depends on the type of signal generated from the hybridization reaction. The most common analysis technique is fluorescent emission from several different types of dyes and fluorophores. However, quantitating samples in this manner usually requires a large amount of the signaling molecule to be present to generate enough emission to be quantitated accurately. More importantly, quantitation of fluorescence generally requires expensive analysis equipment for linear response. Furthermore, the hybridization reactions take up to two hours, which for many uses, such as detecting biological warfare agents, is simply too long. Therefore, a need exists for a system which can rapidly detect biological material in samples.

The present invention is directed to achieving these objectives.

### SUMMARY OF THE INVENTION

The present invention relates to a method of detecting target nucleic acid molecules in a sample. This method involves providing a plurality of different groups of two or more electrically separated electrical conductors with capture probes attached to the conductors such that a gap exists between the capture probes on the electrically separated conductors. The capture probes are contacted with a sample, potentially containing the target nucleic acid molecules, under conditions effective to permit any of the target nucleic acid molecule present in the sample to hybridize to the capture probes and thereby connect the capture probes. The presence of the target nucleic acid molecules is detected by determining whether electricity is conducted between the electrically separated conductors.

The present invention also relates to a device for detecting a target nucleic acid molecule in a sample. The device contains a plurality of groups of two or more electrically separated conductors and capture probes attached to the conductors such that a gap exists between the capture probes on the electrically separated conductors, where the capture probes on the different groups of conductors are the same.

Another aspect of the present invention relates to a device for detecting a target nucleic acid molecule in a sample. The device contains a plurality of groups of two or more electrically separated conductors and capture probes attached to the conductors such that a gap exists between the capture probes on the electrically separated conductors, where the capture probes on at least some of the different groups of conductors are different.

In comparison to other detection systems which require the use of fluorescent or radioactive labels and a long reaction time, the present invention discloses a rapid and economical system for detecting target molecules in a sample.

### BRIEF DESCRIPTION OF THE DRAWINGS

FIG. 1 is a schematic drawing showing one embodiment of the present invention with a plurality of different groups of electrically separated electrical conductors for detection of target nucleic acid molecules.

FIG. 2 is a schematic drawing showing a second embodiment of the present invention with a plurality of different groups of electrically separated electrical conductors for detection of target nucleic acid molecules.

FIG. 3 is a schematic drawing showing a third embodiment of the present invention with a plurality of different groups of electrically separated electrical conductors for detection of target nucleic acid molecules.

FIG. 4 is a schematic drawing showing a fourth embodiment of the present invention with a plurality of different groups of electrically separated electrical conductors for detection of target nucleic acid molecules.

FIGS. 5A-B show a perspective view of a system for detection of a target nucleic acid molecule from a sample which includes a desk-top detection unit and a cartridge which is inserted into the desk-top unit. FIG. 5C shows a schematic view of this system.

FIGS. 6A-B show a perspective view of a system for detection of a target nucleic acid molecule which includes a portable detection unit and a cartridge which is inserted into the portable unit. FIG. 6C shows a schematic view of this system.

### DETAILED DESCRIPTION OF THE INVENTION

The present invention relates to a method of detecting target nucleic acid molecules in a sample. This method involves providing a plurality of different groups of two or more electrically separated electrical conductors with capture probes attached to the conductors such that a gap exists between the capture probes on the electrically separated conductors. The capture probes are contacted with a sample, potentially containing the target nucleic acid molecules, under conditions effective to permit any of the target nucleic acid molecule present in the sample to hybridize to the capture probes and thereby connect the capture probes. The presence of the target nucleic acid molecules is detected by determining whether electricity is conducted between the electrically separated conductors.

One aspect of the present invention involves the detection of multiple DNA sequences from a plurality of DNA sequences based on hybridization techniques. This method involves a sample collection method whereby bacteria, viruses or other DNA containing species are collected and concentrated. This method also incorporates a sample preparation method that involves the liberation of the genetic components. After liberating the DNA, the sample is injected into a detection chip containing complementary DNA probes for the target of interest. In this manner, the device may contain multiple sets of probe molecules that each recognizes a single but different DNA sequence. This process ultimately involves the detection of hybridization products.

In the collection phase, bacteria, viruses or other DNA containing samples are collected and concentrated. A plurality of collection methods will be used depending on the type of sample to be analyzed. Liquid samples will be collected by placing a constant volume of the liquid into a lysis buffer. Airborne samples can be collected by passing air over a filter for a constant time. The filter will be washed with lysis buffer. Alternatively, the filter can be placed directly into the lysis buffer. Waterborne samples can be collected by passing a constant amount of water over a filter. The filter can then be washed with lysis buffer or soaked directly in the lysis buffer. Dry samples can be directly deposited into lysis buffer for removal of the organism of interest.

After sample collection and lysis, cell debris can be removed by precipitation or filtration. Ideally, the sample will be concentrated by filtration, which is more rapid and does not required special reagents. Samples will be forced through filters that will allow only the cellular material to pass through, trapping whole organisms and broken cell debris.

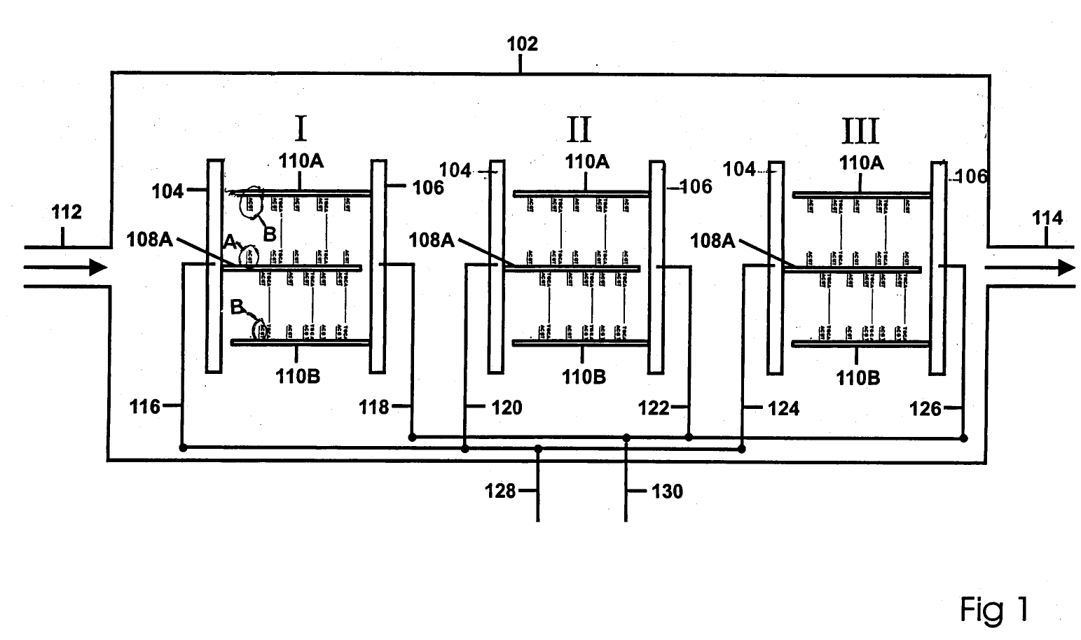

The detection device described in this invention can be as simple as recognizing a single DNA sequence and hence a single organism, or as complex as recognizing multiple DNA sequences. Therefore, different types of devices can be constructed depending on the complexity of the application. FIG. 1 represents a test cartridge 102 that contains an injection port 112 and an exit port 114. Inside the cartridge, are a series of test structures 108A, 110A, and 110B that are initially electrically separated. In one aspect of this invention, the cartridge is designed to recognize a single DNA sequence and, therefore, only two types of probe molecules are attached to separate electrodes probe A to electrode 108 and probe B to electrodes 110A and 110B. In this aspect of the present invention, each test structure group I, II, and III is identical. A plurality of DNA sequences are injected into the cartridge through a port 112. Only the sequences complementary to the probes bound to electrodes will be retained in the cartridge. The unbound DNA sequences exit the cartridge through the exit port 114. Any DNAs that bind to probes A and B will form bridges and complete the connection between electrodes 104 and 106.

Here, the electrical conductivity of nucleic acid molecules is relied upon to transmit the electrical signal. Hans-Werner Fink and Christian Schoenenberger reported in Nature (1999), which is hereby incorporated by reference in its entirety, that DNA conducts electricity like a semiconductor. This flow of current can be sufficient to construct a simple switch, which will indicate whether or not a target nucleic acid molecule is present within a sample. Optionally, after hybridization of the target nucleic acid molecules to sets of capture probes, the nucleic acid molecules can be coated with a conductor, such as a metal, as described in U.S. Patent Application Serial Nos. 60/095,096 or 60/099,506, which are hereby incorporated by reference in their entirety. The coated nucleic acid molecule can then conduct electricity across the gap between the pair of probes, thus producing a detectable signal indicative of the presence of a target nucleic acid molecule. In order to increase the changes of capturing a target DNA of interest from a dilute and complex mixture of DNA sequences, multiple test structures can be placed within the test cartridge. Although FIG. 1 is a representation of 3 similar test structures, many more test structures can be placed within a single cartridge. The advantage of this approach is to be able to overcome a low false positive rate by positively identifying the presence of any organism by use of statistics. In one aspect of this invention, all probe electrical pads 104I, 104II, and 104III are connected to a single but common wire 128 by wires 116, 120, and 124, respectively. Likewise, all electrical pads 106I, 106II, and 106III are connected to a different but common wire 130 by wires 118, 122, and 126, respectively. Therefore, a positive event on any test structure will give a signal from the device. In another aspect of this invention, the groups of electrical pads 104I and 106I, 104II and 106II, and 104III and 106III are separately connected to a power source to permit conduction to be measured separately across each of test structure groups I, II, and III.

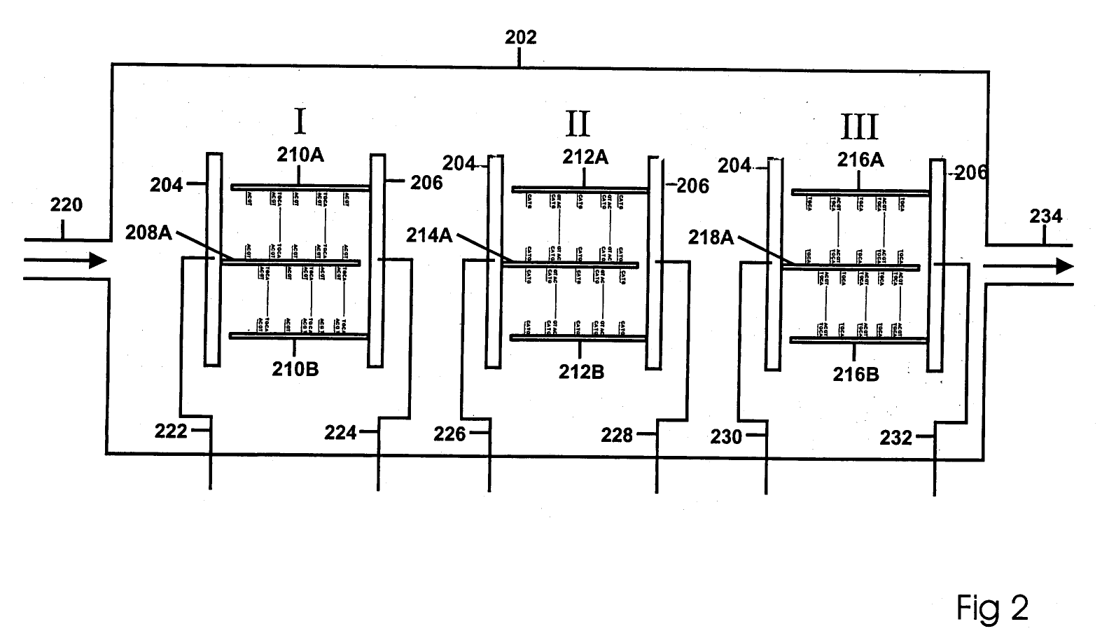

FIG. 2 represents a test cartridge that contains test structure groups I, II, and III whereby each is designed to recognize different DNA sequences. Test structure group I is comprised of two electrical pads 204 and 206 and electrodes 208A, 210A, and 210B attached to each electrical pad that are initially separated. Electrical pads 204 and 206 from each test structure group are each connected to separate wires that connect the cartridge to an analysis device (not shown). For instance, for test structure group I, electrical pads 204 and 206 are connected to wires 222 and 224, respectively. Likewise, in test structure group II, electrical pads 204 and 206 are connected to wires 226 and 228, respectively, while, in test structure group III, electrical pads 204 and 206 are connected to wires 230 and 232, respectively. Test structure groups II and III are identical to test structure group I, except that different sequences of probes are attached to the electrodes. A plurality of DNA sequences is injected into cartridge 202 through a port 220. Only the sequences complementary to the probes bound to electrodes will be retained in the cartridge. The unbound DNA sequences exit the cartridge through the exit port 234. Any DNAs that have bound will form bridges and complete the connection between electrodes 204 and 206. In this aspect of the invention, different DNA sequences can be distinguished by determining the pattern of electrical connections made within the cartridge.

In one aspect of this invention, test structure groups are designed to recognize multiple DNA targets from one organism. Therefore, test structure groups I, II, and III have different probe sequences attached to the electrodes and can recognize multiple target sequences. In another aspect of this invention, test structures are designed to recognize target DNAs from multiple organisms. In the first aspect, test structure groups I, II, and III from FIG. 2 will contain different DNA sequences within a single gene from a single organism, or sequences from multiple genes from a single organism. Since many genes have conserved sequences among family members, designing the invention in this manner allows for a more positive identification of closely related organisms. In the second aspect, this invention can be designed to screen for the presence of multiple organisms. In this example, probe sets are designed and attached to separate test structures to recognize genes from different organisms. Test structures such as those just described could be used for diagnostic purposes.

The second application of the present invention is to obtain quantitative information about the abundance of a target molecule. Some disease states are rated by the number of organisms or molecules present in a fixed quantity of fluid. For example, the severity of HIV diagnosis is often dependent on viral load, or the amount of HIV particles circulating in the bloodstream. Diagnosing any particular patient with HIV is relatively easy to perform, but it would be advantageous to obtain additional quantitative information. Likewise, the progression of the disease and the effectiveness of treatment are again judged by the individual's viral load. In other instances, it would be good to obtain quantitative measurements of the numbers of organisms present in a sample of interest. Not only can the present invention detect the presence of biowarfare agents, such as anthrax or smallpox, but it will allow for precise quantitation of the severity of attack. This is important when determining the level of exposure to personnel, who is to receive antibiotics first, and how best to cleanup and decontaminate after exposure.

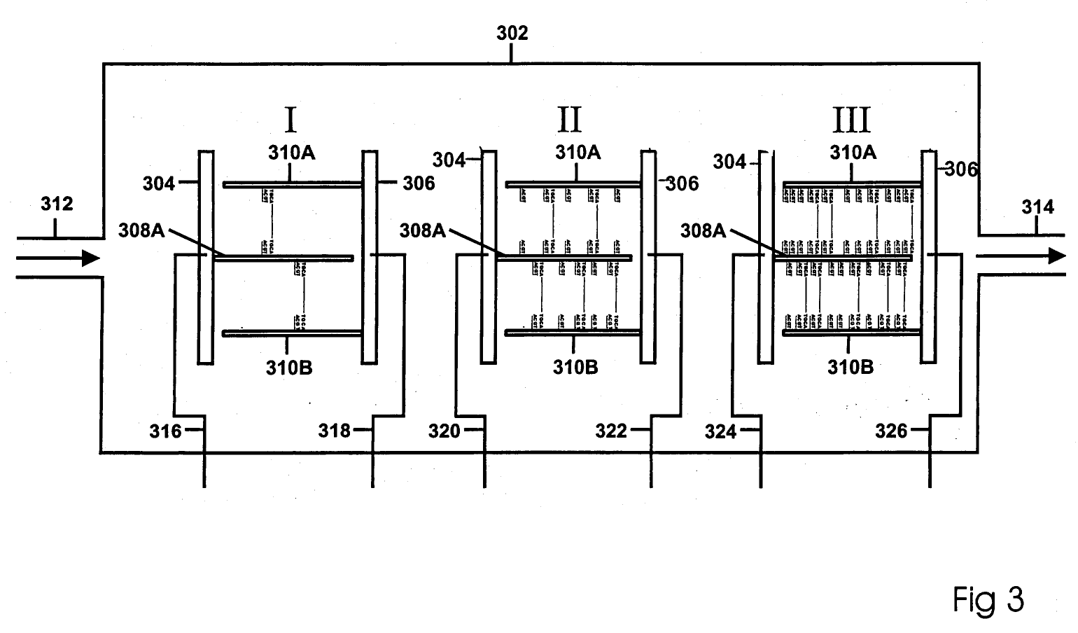

This invention will provide information about the quantity of a contaminant in two aspects. FIG. 3 represents a method of quantitation based on test structure groups containing increasing amounts of probe molecules. The basis for this aspect of this invention is that hybridization occurs in a concentration dependent manner. The greater the number of probe molecules that are bound to an electrode, the greater the chance that a target will bind to that electrode. By limiting the amount of target in a sample, hybridization becomes dependent on the number of probe molecules bound to the electrodes. Therefore, hybridization will occur efficiently on a subset of the electrodes contained within the quantitation cartridge and not at all on electrodes where very few probes have been placed. The quantity of targets present in any sample can then be directly determined by which test structures have formed conductive bridges. The quantitation cartridge 302 in FIG. 3 consists of test structure groups I, II, and III that each contain two electrodes 304 and 306. Smaller electrodes 308A, 310A, and 310B are connected to these electrodes that are initially electrically separated. Each test structure group is connected to an analysis device (not shown) by separate wires 316 to 326 running from one of electrodes 304 and 306 in a particular test group to the outside of the cartridge 302. A plurality of DNA sequences is injected into the cartridge 302 through a port 312. Only the sequences complementary to the probes bound to electrodes will be retained in the cartridge. The unbound DNA sequences exit the cartridge through and exit port 314. Any DNAs that have bound will form bridges and complete the connection between electrodes 304 and 306.

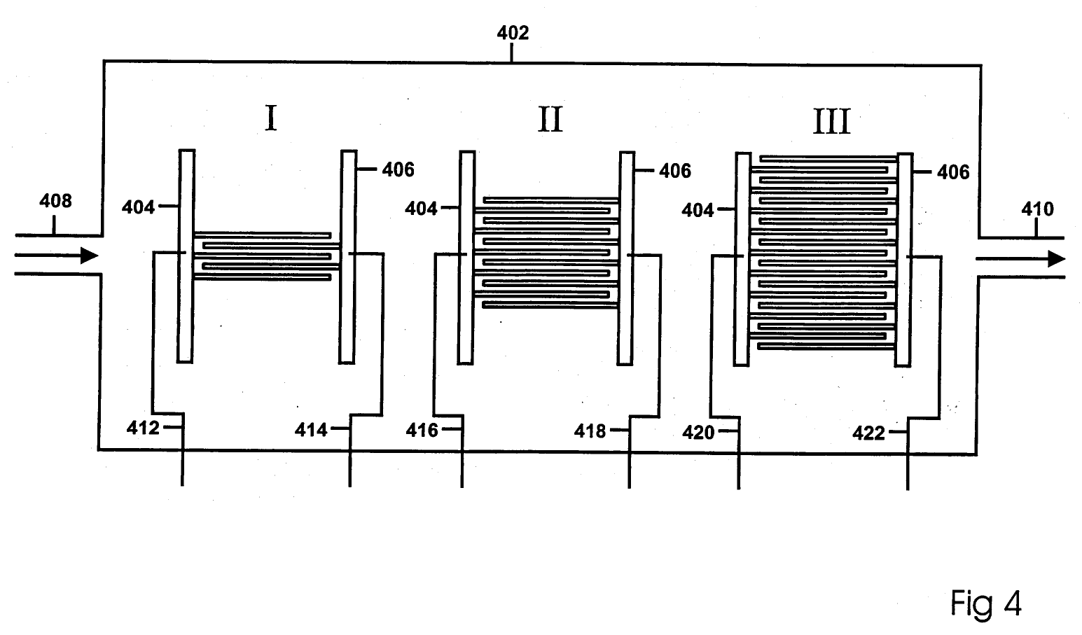

In another aspect of this invention, quantitation may be obtained by constructing test structure groups with increasing numbers of finger projections. FIG. 4 represents a quantitation cartridge 402 whereby test structure groups I, II, and III, respetively, contain 5, 11 and 19 interdigitated finger electrodes. In other aspects of this invention, test structures contain tens, hundreds, or thousands of finger electrodes. Each test structure contains two electrodes 404 and 406 that are initially electrically separated. Each electrode contains a set of interdigitated fingers. The electrodes 404 and 406 are connected to an analysis device (not shown) by individual wires 412 to 422. A plurality of DNA sequences is injected into the cartridge 402 through a port 408. Only the sequences complementary to the probes bound to electrodes will be retained in the cartridge. The unbound DNA sequences exit the cartridge through and exit port 410. Any DNAs that have bound will form bridges and complete the connection between electrodes 404 and 406. In this representation, probe molecules are bound in equal concentrations on the electrodes of each test structure. By increasing the number of electrodes, the number of exposed probe molecules for hybridization effectively increases. As discussed previously, hybridization then becomes dependent on the amount of target present. Quantities of target molecule present in the sample can be determined by the pattern of test structures that contain conductive bridges.

In one aspect of this invention, a positive identification is defined as the detection of a DNA bridge that spans the distance between two sets of electrodes and forms a closed circuit between the two sets of electrodes. As described in this invention, a positive signal can arise by two means. A target DNA complementary to the capture probes can form a bridge and close the circuit, a “real” signal. Inherent in any hybridization assay, some circuits may be formed by the non-specific binding of nucleic acid molecules, not related to the target nucleic acid of interest, so called “false positives”. For any particular test, there will exist a ratio of real to false positive signals that is defined by the complex mixture of nucleic acids passed over the test structures and the particular capture probes bound to the electrodes. It is desirable to keep the false positive signal as low as possible so as to be able to more clearly determine if the specific target nucleic acid is present in the sample. For any particular nucleic acid detection test, it will be necessary to define the “false positive” signal percentage. Once defined, a statistically relevant number of real signals above the false positive signals can be used to determine if a particular nucleic acid is present in a sample.

One aspect of this invention details the ability to multiplex tests for nucleic acid sequences within one test cartridge. It is possible that different statistical criteria will be needed to determine the presence of target nucleic acid if the multiplexing test contains capture probes specific for a single or multiple nucleic acid sequences. In the case of a multiplexing test for a single nucleic acid sequence, one must consider the false positive rate for only one chemical reaction. However, if the multiplexing test contains many different sets of capture probes then each test will have its own false positive rate and each rate must be considered separately. The later case requires a much more rigorous statistical analysis.

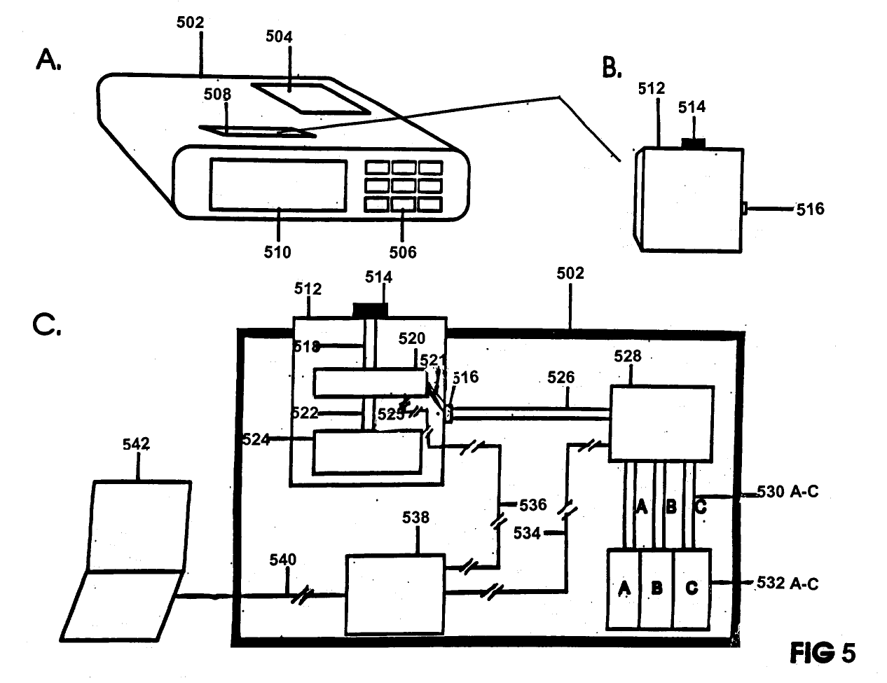

FIGS. 5A-B show a perspective view of a system for detection of a target nucleic acid molecule from a sample. This system includes a desk-top detection unit and a detection cartridge which is inserted into the desk-top unit. In this embodiment, desk-top detection unit 502 is provided with door 504 for filling reagents, control buttons 506, and visual display 510. Slot 508 in desk-top detection unit 502 is configured to receive detection cartridge 512. Detection cartridge 512 further contains first injection port 514 through which a sample solution can be introduced into a first chamber in cartridge 512 and second injection port 516 through which reagents can be introduced into the first chamber.

FIG. 5C shows a schematic view of the system utilizing desk-top detection unit 502 and cartridge 512. In this system, desk-top detection unit 502 contains containers 532A-C suitable for holding reagents and positioned to discharge the reagents into first chamber 520 of detection cartridge 512 through second injection port 516 and conduit 521. Containers 532A-C can, for example, carry a neutralizer, a buffer, a conductive ion solution, and an enhancer. The contents of these containers can be replenished through door 504. This is achieved by making containers 532A-C sealed and disposable or by making them refillable.

Pump 528 removes reagents from containers 532A-C, through tubes 530A-C, respectively, and discharges them through tube 526 and second injection port 516 into detection cartridge 512. Instead of using single pump 528 to draw reagents from containers 532A-C, a separate pump can be provided for each of containers 532A-C so that their contents can be removed individually.

Alternatively, the necessary reagents may be held in containers inside the detection cartridge. The pumps in the detection unit can force a material, such as air, water or oil, into the detection cartridge to force the reagents from the respective containers and into the first chamber. The reagents are then changed with each detection cartridge, which eliminates the buildup of salt precipitates in the detection unit.

Desk-top detection unit 512 is also provided with controller 538, which is in electrical communication with the electrical conductors of the detection cartridge 512 by means of electrical connector 536, to detect the presence of the target molecule in the sample. Controller 538 also operates pump 528 by way of electrical connector 534. Alternatively, separate controllers can be used for operating the pumps and the detection of target molecules. Digital coupling 540 permits controller 538 to communicate data to computer 542 which is external of desk-top detection unit 512.

Detection cartridge 512 contains first chamber 520 which, as noted supra, receives reagents from within desk-top detection unit 502 by way of second injection port 516 and conduit 521. A sample to be analyzed is discharged to first chamber 520 through first injection port 514 and conduit 518. As described more fully with reference to FIGS. 1 to 4 supra, the presence of a target molecule is detected in first chamber 520. Detection cartridge 512 is further provided with second chamber 524 for collecting material discharged from first chamber 520 by way of connector 522. The detection cartridge also contains electrical connector 525 extending through the housing and coupled to the electrically separated conductors in first chamber 520 so that the presence of a target molecule in a sample can be detected.

The detection of a target molecule using a desk-top detection system, as shown in FIGS. 5A-C, can be carried out as follows. After lysis and clarification of the sample, the sample is introduced into detection cartridge 512 through first injection port 514 and conduit 518 and into first chamber 520. Once the sample is introduced, detection cartridge 512 is inserted into slot 508 of desk-top detection unit 502 so that second injection port 516 is connected to conduit 521 and electrical connector 536 is coupled to electrical connector 525. The sample is processed in first chamber 520 containing the capture probes and electrical conductors (like those shown in FIGS. 1-4) for a period of time sufficient for detection of a target nucleic acid molecule in the sample. Processing of the sample within first chamber 520 can involve neutralizing the sample, contacting the neutralized sample with a buffer, then treating the sample with conductive ions, and treating the sample with an enhancer. Molecules that are not captured are expelled from first chamber 520 through second conduit 522 and into second chamber 524. The desk-top detection system can be programmed by a series of operation buttons 506 on the front of the device and the results can be seen on visual display 510.

The detection chip, on which conductive pads and conductive fingers are fixed, is constructed on a support. Examples of useful support materials include, e.g., glass, quartz, and silicon as well as polymeric substrates, e.g. plastics. In the case of conductive or semi-conductive supports, it will generally be desirable to include an insulating layer on the support. However, any solid support which has a non-conductive surface may be used to construct the device. The support surface need not be flat. In fact, the support may be on the walls of a chamber in a chip.

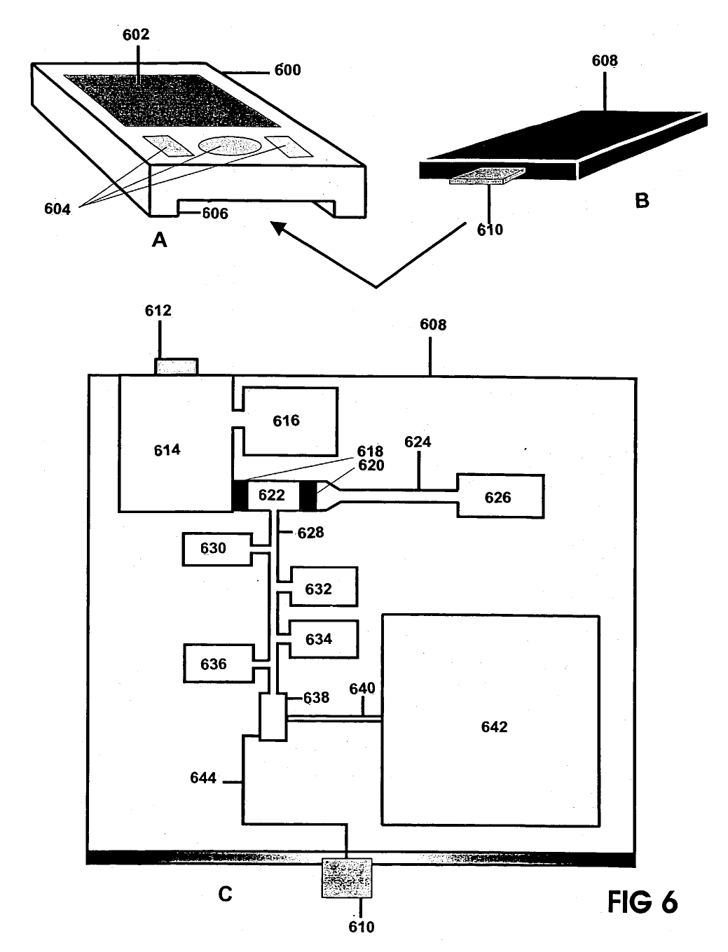

FIGS. 6A-B show a portable detection system. This system is provided with a portable unit 600 which can be in the form of a portable personal digital assistant (e.g., a Palm® unit, 3Com Corporation, Santa Clara, Calif.). Portable unit 600 is provided with visual display 602 and control buttons 604. Slot 606 is provided to receive detection cartridge 608 having electrical connector 610.

FIG. 6C shows a schematic diagram of detection cartridge 608 which is used in the portable detection system of the present invention. Detection cartridge 608 contains first injection port 612 in the housing through which a sample solution can be introduced.

Detection cartridge 608 contains a plurality of containers 630, 632, 634, and 636 suitable for holding reagents and positioned to discharge the reagents into first chamber 638 by way of conduit 628. Containers 630, 632, 634, and 636 can, for example, carry a neutralizer, a buffer, a conductive ion solution, and an enhancer.

Sample pre-treatment chamber 614 is positioned upstream of first chamber 638, and a filter 618 is positioned between pretreatment chamber 614 and first chamber 638. Adjoining pre-treatment chamber 614 is vessel 616 which holds reagents to pre-treat the sample. Detection cartridge 608 also contains pretreatment waste chamber 626 coupled to the pretreatment chamber 614 by way of filter 620 and conduit 624. Second chamber 642 receives material discharged from the first chamber 638 via a connector 640. Detection cartridge 608 includes electrical connector 644 which couples the electrically separated conductors in first chamber 638, like those shown in test cartridges 102, 202, 302, and 402 for the embodiments of FIGS. 1-4, to electrical connector 610.

In operation, the detection of a target molecule using a portable detection system, as shown in FIGS. 6A-C, can be carried out as follows. After lysis and clarification of the sample, the sample solution is introduced into detection cartridge 608 through first injection port 612. Within sample pretreatment chamber 614, the sample can be pretreated with reagents from first container 616. After denaturation and deprotination, the sample can be concentrated by it through filter 618 positioned so that a portion of the pre-treated sample is retained in chamber 622. Excess fluids and unwanted cellular material are passed through filter 620 and waste tube 624 and are collected in pretreatment waste chamber 626. The portion of the sample solution which passes to first chamber 638 is neutralized by the addition of a neutralizer from second container 630. Within first chamber 638, the neutralized target nucleic acid molecule, if present in the sample, is permitted to hybridize with the capture probes on the detection chip in first chamber 638 in substantially the same way as described above with reference to FIGS. 1 to 4. During this period, the contents of first chamber 638 are contacted with a buffer from third container 632. After binding and washing, the sample is treated with a conductive ion solution from fourth container 634, such that conductive ions are deposited on the target molecules that have hybridized to the capture probes on the detection chip. Additionally, after treatment with a conductive ion solution, the sample can be treated with an enhancer solution from fifth container 636 to grow a continuous layer of conductive metal from the deposited conductive ions. Excess buffers and waste buffers will exit first chamber 638 through waste tube 640 and collect in second chamber 642. Electrical connector 644 couples the electrically separated conductors on the detection chip to electrical connector 610 which is connected to portable unit 600. The portable detection system can be programmed by operation of a series of buttons 604 on the front of portable unit 600, and the results are visualized on screen 602.

In carrying out the method of the present invention, a sample collection phase is initially carried out where bacteria, viruses, or other species are collected and concentrated. The target nucleic acid molecule whose sequence is to be determined is usually isolated from a tissue sample. If the target nucleic acid molecule is genomic, the sample may be from any tissue (except exclusively red blood cells). For example, whole blood, peripheral blood lymphocytes or PBMC, skin, hair, or semen are convenient sources of clinical samples. These sources are also suitable if the target is RNA. Blood and other body fluids are also a convenient source for isolating viral nucleic acids. If the target nucleic acid molecule is mRNA, the sample is obtained from a tissue in which the mRNA is expressed. If the target nucleic acid molecule in the sample is RNA, it may be reverse transcribed to DNA, but need not be converted to DNA in the present invention.

Further details of how to carry out the process of the present invention are set forth in U.S. Pat. No. 6,399,303 B1 to Connolly, which is hereby incorporated by reference in its entirety.

A plurality of collection methods can be used depending on the type of sample to be analyzed. Liquid samples can be collected by placing a constant volume of the liquid into a lysis buffer. Airborne samples can be collected by passing air over a filter for a constant time. The filter can be washed with lysis buffer. Alternatively, the filter can be placed directly into the lysis buffer. Waterborne samples can be collected by passing a constant amount of water over a filter. The filter can then be washed with lysis buffer or soaked directly in the lysis buffer. Dry samples can be directly deposited into lysis buffer for removal of the organism of interest.

When whole cells, viruses, or other tissue samples are being analyzed, it is typically necessary to extract the nucleic acids from the cells or viruses, prior to continuing with the various sample preparation operations. Accordingly, following sample collection, nucleic acids may be liberated from the collected cells, viral coat, etc., into a crude extract, followed by additional treatments to prepare the sample for subsequent operations such as denaturation of contaminating (DNA binding) proteins, purification, filtration, and desalting.

Liberation of nucleic acids from the sample cells or viruses, and denaturation of DNA binding proteins may generally be performed by physical or chemical methods. For example, chemical methods generally employ lysing agents to disrupt the cells and extract the nucleic acids from the cells, followed by treatment of the extract with chaotropic salts such as guanidinium isothiocyanate or urea, to denature any contaminating and potentially interfering proteins. Generally, where chemical extraction and/or denaturation methods are used, the appropriate reagents may be incorporated within the extraction chamber, a separate accessible chamber, or externally introduced.

Alternatively, physical methods may be used to extract the nucleic acids and denature DNA binding proteins. U.S. Pat. No. 5,304,487, which is hereby incorporated by reference in its entirety, discusses the use of physical protrusions within microchannels or sharp edged particles within a chamber or channel to pierce cell membranes and extract their contents. More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current to the sample. More specifically, the sample of cells is flowed through a microtubular array while an alternating electric current is applied across the fluid flow. A variety of other methods may be utilized within the device of the present invention to effect cell lysis/extraction, including, e.g., subjecting cells to ultrasonic agitation, or forcing cells through microgeometry apertures, thereby subjecting the cells to high shear stress resulting in rupture.

Following extraction, it is often desirable to separate the nucleic acids from other elements of the crude extract, e.g., denatured proteins, cell membrane particles, and the like. Removal of particulate matter is generally accomplished by filtration, flocculation, or the like. Ideally, the sample is concentrated by filtration, which is more rapid and does not require special reagents. A variety of filter types may be readily incorporated into the device. Samples can be forced through filters that will allow only the cellular material to pass through, trapping whole organisms and broken cell debris. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample. Suitable solid supports for nucleic acid binding include, e.g., diatomaceous earth, silica, or the like. Suitable gel exclusion media is also well known in the art and is commercially available from, e.g., Pharmacia and Sigma Chemical. This isolation and/or gel filtration/desalting may be carried out in an additional chamber, or alternatively, the particular chromatographic media may be incorporated in a channel or fluid passage leading to a subsequent reaction chamber.

The probes are preferably selected to bind with the target such that they have approximately the same melting temperature. This can be done by varying the lengths of the hybridization region. A-T rich regions may have longer target sequences, whereas G-C rich regions would have shorter target sequences.

Hybridization assays on substrate-bound oligonucleotide arrays involve a hybridization step and a detection step. In the hybridization step, the sample potentially containing the target and an isostabilizing agent, denaturing agent, or renaturation accelerant is brought into contact with the probes of the array and incubated at a temperature and for a time appropriate to allow hybridization between the target and any complementary probes.

Including a hybridization optimizing agent in the hybridization mixture significantly improves signal discrimination between perfectly matched targets and single-base mismatches. As used herein, the term “hybridization optimizing agent” refers to a composition that decreases hybridization between mismatched nucleic acid molecules, i.e., nucleic acid molecules whose sequences are not exactly complementary.

An isostabilizing agent is a composition that reduces the base-pair composition dependence of DNA thermal melting transitions. More particularly, the term refers to compounds that, in proper concentration, result in a differential melting temperature of no more than about 1° C. for double stranded DNA oligonucleotides composed of AT or GC, respectively. Isostabilizing agents preferably are used at a concentration between 1 M and 10 M, more preferably between 2 M and 6 M, most preferably between 4 M and 6 M, between 4 M and 10 M, and, optimally, at about 5 M. For example, a 5 M agent in 2×SSPE (Sodium Chloride/Sodium Phosphate/EDTA solution) is suitable. Betaines and lower tetraalkyl ammonium salts are examples of suitable isostabilizing agents.

Betaine (N,N,N,-trimethylglycine; (Rees et al., Biochem., (1993) 32:137-144), which is hereby incorporated by reference in its entirety) can eliminate the base pair composition dependence of DNA thermal stability. Unlike tetramethylammonium chloride (“TMACI”), betaine is zwitterionic at neutral pH and does not alter the polyelectrolyte behavior of nucleic acids while it does alter the composition-dependent stability of nucleic acids. Inclusion of betaine at about 5 M can lower the average hybridization signal, but increases the discrimination between matched and mismatched probes.

A denaturing agent is a compositions that lowers the melting temperature of double stranded nucleic acid molecules by interfering with hydrogen bonding between bases in a double-stranded nucleic acid or the hydration of nucleic acid molecules. Denaturing agents can be included in hybridization buffers at concentrations of about 1 M to about 6 M and, preferably, about 3 M to about 5.5 M.

Denaturing agents include formamide, formaldehyde, dimethylsulfoxide (“DMSO”), tetraethyl acetate, urea, guanidine thiocyanate (“GuSCN”), glycerol and chaotropic salts. As used herein, the term “chaotropic salt” refers to salts that function to disrupt van der Waal's attractions between atoms in nucleic acid molecules. Chaotropic salts include, for example, sodium trifluoroacetate, sodium tricholoroacetate, sodium perchlorate, and potassium thiocyanate.

A renaturation accelerant is a compound that increases the speed of renaturation of nucleic acids by at least 100-fold. They generally have relatively unstructured polymeric domains that weakly associate with nucleic acid molecules. Accelerants include heterogenous nuclear ribonucleoprotein (“hnRP”) A1 and cationic detergents such as, preferably, cetyltrimethylammonium bromide (“CTAB”) and dodecyl trimethylammonium bromide (“DTAB”), and, also, polylysine, spermine, spermidine, single stranded binding protein (“SSB”), phage T4 gene 32 protein, and a mixture of ammonium acetate and ethanol. Renaturation accelerants can be included in hybridization mixtures at concentrations of about 1 μM to about 10 mM and, preferably, 1 μM to about 1 mM. The CTAB buffers work well at concentrations as low as 0.1 mM.

Addition of small amounts of ionic detergents (such as N-lauroylsarkosine) to the hybridization buffers can also be useful. LiCl is preferred to NaCl. Hybridization can be at 20°-65° C., usually 37° C. to 45° C. for probes of about 14 nucleotides. Additional examples of hybridization conditions are provided in several sources, including: Sambrook et al., Molecular Cloning: A Laboratory Manual, 2nd Ed., Cold Spring Harbor, N.Y. (1989); and Berger and Kimmel, “Guide to Molecular Cloning Techniques,” Methods in Enzymology, Volume 152, Academic Press, Inc., San Diego, Calif. (1987); Young and Davis, Proc. Natl. Acad. Sci. USA, 80:1194 (1983), which are hereby incorporated by reference in their entirety.

In addition to aqueous buffers, non-aqueous buffers may also be used. In particular, non-aqueous buffers which facilitate hybridization but have low electrical conductivity are preferred.

The sample and hybridization reagents are placed in contact with the array and incubated. Contact can take place in any suitable container, for example, a dish or a cell specially designed to hold the probe array and to allow introduction and removal of fluids. Generally, incubation will be at temperatures normally used for hybridization of nucleic acids, for example, between about 20° C. and about 75° C., e.g., about 25° C., about 30° C., about 35° C., about 40° C., about 45° C., about 50° C., about 55° C, about 60° C., or about 65° C. For probes longer than about 14 nucleotides, 37-45° C. is preferred. For shorter probes, 55-65° C. is preferred. More specific hybridization conditions can be calculated using formulae for determining the melting point of the hybridized region. Preferably, hybridization is carried out at a temperature at or between ten degrees below the melting temperature and the melting temperature. More preferred, hybridization is carried out at a temperature at or between five degrees below the melting temperature and the melting temperature. The target is incubated with the capture probes for a time sufficient to allow the desired level of hybridization between the target and any complementary capture probes. After incubation with the hybridization mixture, the electrically separated conductors are washed with the hybridization buffer, which also can include the hybridization optimizing agent. These agents can be included in the same range of amounts as for the hybridization step, or they can be eliminated altogether.

Details on how capture probes are attached to electrical conductors are set forth in U.S. patent application Ser. No. 10/288,657, which is hereby incorporated by reference in its entirety.

Various other methods exist for attaching the capture probes to the electrical conductors. For example, U.S. Pat. Nos. 5,861,242, 5,861,242, 5,856,174, 5,856,101, and 5,837,832, which are hereby incorporated by reference in their entirety, disclose a method where light is shone through a mask to activate functional (for oligonucleotides, typically an —OH) groups protected with a photo-removable protecting group on a surface of a solid support. After light activation, a nucleoside building block, itself protected with a photo-removable protecting group (at the 5′-OH), is coupled to the activated areas of the support. The process can be repeated, using different masks or mask orientations and building blocks, to place probes on a substrate.

Alternatively, new methods for the combinatorial chemical synthesis of peptide, polycarbamate, and oligonucleotide arrays have recently been reported (see Fodor et al., Science, 251:767-773 (1991); Cho et al., Science, 261:1303-1305 (1993); and Southern et al., Genomics 13:1008-10017 (1992), which are hereby incorporated by reference in their entirety). These arrays (see Fodor et al., Nature, 364:555-556 (1993), which is hereby incorporated by reference in its entirety) harbor specific chemical compounds at precise locations in a high-density, information rich format, and are a powerful tool for the study of biological recognition processes.

Preferably, the probes are attached to the leads through spatially directed oligonucleotide synthesis. Spatially directed oligonucleotide synthesis may be carried out by any method of directing the synthesis of an oligonucleotide to a specific location on a substrate. Methods for spatially directed oligonucleotide synthesis include, without limitation, light-directed oligonucleotide synthesis, microlithography, application by ink jet, microchannel deposition to specific locations and sequestration with physical barriers. In general, these methods involve generating active sites, usually by removing protective groups, and coupling to the active site a nucleotide which, itself, optionally has a protected active site if further nucleotide coupling is desired.

In one embodiment, the lead-bound oligonucleotides are synthesized at specific locations by light-directed oligonucleotide synthesis which is disclosed in U.S. Pat. No. 5,143,854, Published PCT application Ser. No. WO 92/10092, and Published PCT application Ser. No. WO 90/15070, which are hereby incorporated by reference in their entirety. In a basic strategy of this process, the surface of a solid support modified with linkers and photolabile protecting groups is illuminated through a photolithographic mask, yielding reactive hydroxyl groups in the illuminated regions. A 3′-O-phosphoramidite-activated deoxynucleoside (protected at the 5′-hydroxyl with a photolabile group) is then presented to the surface and coupling occurs at sites that were exposed to light. Following the optional capping of unreacted active sites and oxidation, the substrate is rinsed and the surface is illuminated through a second mask, to expose additional hydroxyl groups for coupling to the linker. A second 5′-protected, 3′O-phosphoramidite-activated deoxynucleoside (C-X) is presented to the surface. The selective photodeprotection and coupling cycles are repeated until the desired set of probes are obtained. Photolabile groups are then optionally removed, and the sequence is, thereafter, optionally capped. Side chain protective groups, if present, are also removed. Since photolithography is used, the process can be miniaturized to specifically target leads in high densities on the support.

The protective groups can, themselves, be photolabile. Alternatively, the protective groups can be labile under certain chemical conditions, e.g., acid. In this example, the surface of the solid support can contain a composition that generates acids upon exposure to light. Thus, exposure of a region of the substrate to light generates acids in that region that remove the protective groups in the exposed region. Also, the synthesis method can use 3′-protected 5′-O-phosphoramidite-activated deoxynucleoside. In this case, the oligonucleotide is synthesized in the 5′ to 3′ direction, which results in a free 5′ end.

The general process of removing protective groups by exposure to light, coupling nucleotides (optionally competent for further coupling) to the exposed active sites, and optionally capping unreacted sites is referred to herein as “light-directed nucleotide coupling.”

The probes may be targeted to the electrically separated conductors by using a chemical reaction for attaching the probe or nucleotide to the conductor which preferably binds the probe or nucleotide to the conductor rather than the support material. Alternatively, the probe or nucleotide may be targeted to the conductor by building up a charge on the conductor which electrostatically attracts the probe or nucleotide.

Nucleases can be used to remove probes which are attached to the wrong conductor. More particularly, a target nucleic acid molecule may be added to the probes. Targets which bind at both ends to probes, one end to each conductor, will have no free ends and will be resistant to exonuclease digestion. However, probes which are positioned so that the target cannot contact both conductors will be bound at only one end, leaving the molecule subject to digestion. Thus, improperly located probes can be removed while protecting the properly located probes. After the protease is removed or inactivated, the target nucleic acid molecule can be removed and the device is ready for use.

The capture probes can be formed from natural nucleotides, chemically modified nucleotides, or nucleotide analogs, as long as they have activated hydroxyl groups compatible with the linking chemistry. Such RNA or DNA analogs comprise but are not limited to 2′-O-alkyl sugar modifications, methylphosphonate, phosphorothioate, phosphorodithioate, formacetal, 3′-thioformacetal, sulfone, sulfamate, and nitroxide backbone modifications, amides, and analogs, where the base moieties have been modified. In addition, analogs of oligomers may be polymers in which the sugar moiety has been modified or replaced by another suitable moiety, resulting in polymers which include, but are not limited to, polyvinyl backbones (Pitha et al., “Preparation and Properties of Poly (I-vinylcytosine),” Biochim. Biophys. Acta, 204:381-8 (1970); Pitha et al., “Poly(1-vinyluracil): The Preparation and Interactions with Adenosine Derivatives,” Biochim. Biophys. Acta, 204:39-48 (1970), which are hereby incorporated by reference in their entirety), morpholino backbones (Summerton, et al., “Morpholino Antisense Oligomers: Design, Preparation, and Properties,” Antisense Nucleic Acid Drug Dev., 7:187-9 (1997), which is hereby incorporated by reference in its entirety) and peptide nucleic acid (PNA) analogs (Stein et al., “A Specificity Comparison of Four Antisense Types: Morpholino, 2′-O-methyl RNA, DNA, and Phosphorothioate DNA,” J. Antisense Nucleic Acid Drug Dev., 7:151-7 (1997); Egholm et al., “Peptide Nucleic Acids (PNA)-Oligonucleotide Analogues with an Achiral Peptide Backbone,” (1992); Faruqi et al., “Peptide Nucleic Acid-Targeted Mutagenesis of a Chromosomal Gene in Mouse Cells,” Proc. Natl. Acad. Sci. USA, 95:1398-403 (1998); Christensen et al., “Solid-Phase Synthesis of Peptide Nucleic Acids,” J. Pept. Sci., 1:175-83 (1995); Nielsen et al., “Peptide Nucleic Acid (PNA). A DNA Mimic with a Peptide Backbone,” Bioconjug. Chem., 5:3-7 (1994), which are hereby incorporated by reference in their entirety).

The capture probes can contain the following exemplary modifications: pendant moieties, such as, proteins (including, for example, nucleases, toxins, antibodies, signal peptides and poly-L-lysine); intercalators (e.g., acridine and psoralen), chelators (e.g., metals, radioactive metals, boron and oxidative metals), alkylators, and other modified linkages (e.g., alpha anomeric nucleic acids). Such analogs include various combinations of the above-mentioned modifications involving linkage groups and/or structural modifications of the sugar or base for the purpose of improving RNAseH-mediated destruction of the targeted RNA, binding affinity, nuclease resistance, and or target specificity.

The present invention can be used for numerous applications, such as detection of pathogens. For example, samples may be isolated from drinking water or food and rapidly screened for infectious organisms. The present invention may also be used for food and water testing. In recent times, there have been several large recalls of tainted meat products. The detection system of the present invention can be used for the in-process detection of pathogens in foods and the subsequent disposal of the contaminated materials. This could significantly improve food safety, prevent food borne illnesses and death, and avoid costly recalls. Capture probes that can identify common food borne pathogens, such as Salmonella and E. coli., could be designed for use within the food industry.

In yet another embodiment, the present invention can be used for real time detection of biological warfare agents. With the recent concerns of the use of biological weapons in a theater of war and in terrorist attacks, the device could be configured into a personal sensor for the combat soldier or into a remote sensor for advanced warnings of a biological threat. The devices which can be used to specifically identify the agent, can be coupled with a modem to send the information to another location. Mobile devices may also include a global positioning system to provide both location and pathogen information.

In yet another embodiment, the present invention may be used to identify an individual. A series of probes, of sufficient number to distinguish individuals with a high degree of reliability, are placed within the device. Various polymorphism sites are used. Preferentially, the device can determine the identity to a specificity of greater than one in one million, more preferred is a specificity of greater than one in one billion, even more preferred is a specificity of greater than one in ten billion.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of detecting target nucleic acid molecules in a sample, said method comprising:
providing a plurality of different groups of two or more electrically separated electrical conductors with capture probes attached to the conductors such that a gap exists between the capture probes on the electrically separated conductors;
contacting the capture probes with a sample, potentially containing the target nucleic acid molecules, under conditions effective to permit any of the target nucleic acid molecule present in the sample to hybridize to the capture probes and thereby connect the capture probes; and
detecting the presence of the target nucleic acid molecules by determining whether electricity is conducted between the electrically separated conductors.

2. The method according to claim 1, wherein the capture probes are oligonucleotides.

3. The method according to claim 1, wherein the capture probes are peptide nucleic acid analogs.

4. The method according to claim 1, wherein the target nucleic acid molecules are DNA.

5. The method according to claim 1, wherein the target nucleic acid molecules are RNA.

6. The method according to claim 1, wherein the capture probes are complementary to target nucleic acid molecules from the genetic material of a pathogenic bacteria.

7. The method according to claim 6, wherein the pathogenic bacteria is a biowarfare agent.

8. The method according to claim 6, wherein the pathogenic bacteria is a foodborne pathogen.

9. The method according to claim 1, wherein the capture probes are complementary to target nucleic acid molecules from the genetic material of a virus.

10. The method according to claim 1, wherein the capture probes are complementary to target nucleic acid molecules from the genetic material of a human.

11. The method according to claim 1, wherein the capture probes are complementary to polymorphisms where the base or bases complementary to the polymorphism are located at an end of the capture probes.

12. The method according to claim 1, wherein the capture probes for a plurality of the different groups of two or more electrically separated electrical conductors are the same so that the same target nucleic acid molecule hybridizes to the capture probes.

13. The method according to claim 12 further comprising:
quantifying the amount of the target nucleic acid molecule in the sample as a result of said detecting the presence of the target nucleic acid molecules by determining whether electricity is conducted between the electrically separated conductors.

14. The method according to claim 13, wherein said quantifying is carried out by having different amounts of the capture probes available for contact with the target nucleic acid molecule on different groups of the two or more electrically separated electrical conductors.

15. The method according to claim 14, wherein the amount of the capture probes available for contact with the target nucleic acid is varied by having the different groups of the two or more electrically separated electrical conductors have different exposed areas.

16. The method according to claim 15, wherein the different groups of the two or more electrically separated electrical conductors have different numbers of the electrically separated electrical conductors.

17. The method according to claim 14, wherein the amount of the capture probes available for contact with the target nucleic acid is varied by having the different groups of the two or more electrically separated electrical conductors have different concentrations of capture probes in a given exposed area.

18. The method according to claim 1, wherein the capture probes for at least some of the different groups of two or more electrically separated electrical conductors are different so that different target nucleic acid molecules hybridize to the capture probes.

19. The method according to claim 18, wherein the different target nucleic acid molecules are from the same source.

20. The method according to claim 19, wherein the source is a pathogenic bacteria.

21. The method according to claim 19, wherein the source is a virus.

22. The method according to claim 19, wherein the source is a human.

23. The method according to claim 18, wherein the different target nucleic acid molecules are from a different source.

24. The method according to claim 23, wherein at least one of the sources is a pathogenic bacteria.

25. The method according to claim 23, wherein at least one of the sources is a virus.

26. The method according to claim 23, wherein at least one of the sources is a human.

27. The method according to claim 18, wherein the capture probes for at least some of the plurality of the different groups of two or more electrically separated electrical conductors are the same so that the same target nucleic acid molecule hybridizes to those same capture probes.

28. The method according to claim 27 further comprising:
quantifying the amount of the target nucleic acid molecule in the sample as a result of said detecting the presence of the target nucleic acid molecules by determining whether electricity is conducted between the electrically separated conductors having the same capture probes.

29. The method according to claim 28, wherein said quantifying is carried out by having different amounts of the capture probes available for contact with the target nucleic acid molecule on different groups of the two or more electrically separated electrical conductors.

30. The method according to claim 29, wherein the amount of the capture probes available for contact with the target nucleic acid is varied by having the different groups of the two or more electrically separated electrical conductors have different exposed areas.

31. The method according to claim 30, wherein the different groups of the two or more electrically separated electrical conductors have different numbers of the electrically separated electrical conductors.

32. The method according to claim 28, wherein the amount of the capture probes available for contact with the target nucleic acid is varied by having the different groups of the two or more electrically separated electrical conductors have different concentrations of capture probes in a given exposed area.

33. The method according to claim 1 further comprising:
coating the capture probes as well as any target nucleic acid molecule hybridized to the capture probe with a conductive material.

34. The method according to claim 33, wherein the conductive material is silver.

35. The method according to claim 33, wherein the conductive material is gold.

36. The method according to claim 1 further comprising:
statistically analyzing results of said detecting.

37. A device for detecting a target nucleic acid molecule in a sample, said device comprising:
a plurality of groups of two or more electrically separated conductors and
capture probes attached to the conductors such that a gap exists between the capture probes on the electrically separated conductors, wherein the capture probes on the different groups of conductors are the same.

38. The device according to claim 37, wherein the device has different amounts of the capture probes available for contact with the target nucleic acid molecule on different groups of the two or more electrically separated electrical conductors.

39. The device according to claim 38, wherein the amount of capture probes available for contact with the target nucleic acid molecule is varied by having said device have different groups of the two or more electrically separated electrical conductors with different exposed areas.

40. The device according to claim 39, wherein the different groups of the two or more electrically separated electrical conductors have different numbers of the electrically separated electrical conductors.

41. The device according to claim 38, wherein the amount of the capture probes available for contact with the target nucleic acid is varied by having the different groups of the two or more electrically separated electrical conductors have different concentrations of capture probes in a given exposed area.

42. The device according to claim 37, wherein the capture probes are oligonucleotides.

43. The device according to claim 37, wherein the capture probes are peptide nucleic acid analogs.

44. A device for detecting a target nucleic acid molecule in a sample, said device comprising:
a plurality of groups of two or more electrically separated conductors and
capture probes attached to the conductors such that a gap exists between the capture probes on the electrically separated conductors, wherein the capture probes on at least some of the different groups of conductors are different.

45. The device according to claim 44, wherein the capture probes for at least some of the plurality of the different groups of conductors are the same so that the same target nucleic acid molecule hybridizes to those same capture probes.

46. The device according to claim 45, wherein the device has different amounts of the capture probes available for contact with the target nucleic acid molecule that are the same on different groups of the two or more electrically separated electrical conductors.

47. The device according to claim 46, wherein the amount of capture probes available for contact with the target nucleic acid molecule is varied by having said device have different groups of the two or more electrically separated electrical conductors with different exposed areas.

48. The device according to claim 47, wherein the different groups of the two or more electrically separated electrical conductors have different numbers of the electrically separated electrical conductors.

49. The device according to claim 46, wherein the amount of the capture probes available for contact with the target nucleic acid is varied by having the different groups of the two or more electrically separated electrical conductors have different concentrations of capture probes in a given exposed area.

50. The device according to claim 44, wherein the capture probes are oligonucleotides.

51. The device according to claim 44, wherein the capture probes are peptide nucleic acid analogs.
